(19) United States
(12) Patent Application Publication
(10) Pub. No.: US 2010/0094138 A1
(43) Pub. Date: Apr. 15, 2010

(54) Imaging catheter using laser profile for plaque depth measurement

(76) Inventors: Morteza Gharib, San Marino, CA (US); Jian Lu, Laguna Hills, CA (US); David Jeon, Pasadena, CA (US)

Correspondence Address: TOPE-MCKAY & ASSOCIATES;CARY TOPE-MCKAY, 23852 Pacific Coast Highway #311, Malibu, CA, 90265, US

(21) Appl. No.: 12/460,988

(22) Filed: Jul. 27, 2009

Related U.S. Application Data
(60) Provisional application No. 61/135,930, filed on Jul. 25, 2008.

(51) Int. Cl. A61B 6/00

(52) U.S. Cl. 600/478

(57) ABSTRACT

A device, system, and method for measuring the depth of a material layer such as a blood vessel plaque layer is disclosed. A fiber optic bundle housed in a balloon catheter projects a laser dot toward a conical mirror, which reflects the dot perpendicularly onto the surface of the plaque. The laser dot is reflected back from the plaque layer with a substantially Gaussian intensity profile. The conical mirror directs the reflected image back to the fiber optic bundle, which delivers the image to a sensor. The depth of the plaque layer can be determined by comparing the diameter of the image intensity profile to a pre-obtained normalized data set.

### PRIORITY CLAIM

The present application is a non-provisional patent application, claiming the benefit of priority of U.S. Provisional Application No. 61/135,930, filed on Jul. 25, 2008, entitled, “IMAGING CATHETER USING LASER PROFILE FOR PLAQUE DEPTH MEASUREMENT.”

### FIELD OF INVENTION

The present invention relates to a catheter imaging system and, more specifically, to a catheter imaging system which uses the Gaussian profile of projected laser dots to determine plaque depth in blood vessels.

### BACKGROUND OF INVENTION

Blood vessel diseases such as atherosclerosis are usually caused by progressive accumulation of plaque, including fat and cholesterol, on the inner vessel walls. Balloon imaging catheters are widely used as a minimally invasive tool for diagnostics or treatment of blood vessel diseases. The thickness of deposited plaque characterizes the seriousness of the disease. Therefore, having an accurate depth measurement of the plaque will provide useful information for diagnostics and in turn significantly enhance the effects of medical treatments. While current imaging systems inside a balloon catheter can obtain planar information regarding the surrounding vessel, measuring the depth of fat and cholesterol deposits along the vessel walls remains a challenge.

Therefore, a continuing need exists for a catheter imaging system which can measure the depth of plaque deposits along blood vessel walls.

### SUMMARY OF INVENTION

The present invention relates to a catheter imaging system and, more specifically, to a catheter imaging system which uses the Gaussian profile of projected laser dots to determine plaque depth in blood vessels. In one aspect, the present invention is a device comprising a fiber optic bundle, a mirror, and a sensor. The fiber optic bundle extends along an axis and comprises a projection portion and a receiving portion. The projection portion is configured to project light onto a material layer surface. The receiving portion is configured to receive a reflected image signal of the projected light from the material layer. A mirror is positioned at a terminus of the fiber optic bundle. The mirror is configured to reflect projected light from the projection portion of the fiber optic bundle at an angle substantially perpendicular to the axis of the fiber optic bundle to illuminate the material surface. The mirror is further configured to reflect the reflected image signal of the projected light from the material layer at an angle substantially perpendicular to the axis of the fiber optic bundle, such that the reflected image signal can be received by the receiving portion of the fiber optic bundle. The sensor is configured to receive the reflected image signal from the receiving portion of the fiber optic bundle, whereby the image signal can be analyzed to determine the depth of the material layer.

In another aspect of the device, the fiber optic bundle is mounted within a balloon catheter.

In yet another aspect, the device is configured to move axially within the balloon catheter.

In a further aspect of the device, the conical mirror is held by a holder portion near a center of the fiber optic bundle.

In yet another aspect of the device, the mirror is substantially conical in shape, and positioned such that an apex of the conical mirror is located proximal to the terminus of the fiber optic bundle.

Another aspect of the present invention is a method for determining a depth of a material layer. The method comprises a first act of projecting light onto a surface of the material layer. Next, a reflected image signal of the projected light is received from the material layer surface. The reflected image signal is then with a sensor. An image intensity profile of the captured image is measured. Typically the measurement is a diameter of the image intensity profile. From this measurement, the depth of the material layer can be determined by comparison of the measured image intensity profile with a pre-obtained normalized data set.

In another aspect of the method of the present invention, the acts of projecting, receiving, and capturing are executed using a catheter imaging system consistent with the device of the present invention, as previously described.

As can be appreciated by one skilled in the art, another aspect of the present invention is a data processing system for measuring the depth of a material layer, comprising one or more processors configured to perform operations of the method of the present invention, as previously described.

In another aspect, the operations of projecting, receiving, and capturing are executed using a catheter imaging system consistent with the device of the present invention, as previously described.

In yet another aspect, as can be appreciated by one skilled in the art, the present invention comprises a computer program product, comprising computer instruction means encoded on a computer-readable medium executable by a computer having a processor for causing the processor to perform the operations of the method of the present invention, as previously described.

In another aspect of the computer program product, the operations of projecting, receiving, and capturing are executed using a catheter imaging system consistent with the device of the present invention, as previously described.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 is an illustration showing the device of the present invention situated within a blood vessel;

FIG. 2A is a graph showing an image intensity profile for a sample of 2.2 mm chicken fat;

FIG. 2B is a graph showing an image intensity profile for a sample of 4.4 mm chicken fat;

FIG. 3 is a graph comparing normalized Gaussian profile of image intensity vs. depth of material layer;

FIG. 4 is a flow diagram showing the acts in the method of the present invention;

FIG. 5 is a block diagram showing the components of a data processing system in accordance with the present invention; and

FIG. 6 is an illustration showing computer program products in accordance with the present invention.

### DETAILED DESCRIPTION

The present invention relates to a catheter imaging system and, more specifically, to a catheter imaging system which uses the Gaussian profile of projected laser dots to determine plaque depth in blood vessels. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

(1) Description

The present invention relates to a catheter imaging system and, more specifically, to a catheter imaging system which uses the Gaussian profile of projected laser dots to determine plaque depth in blood vessels. It has been observed that when a narrow band laser beam shines on a plaque surface, both the Gaussian profile and the intensity profile of the laser dot vary depending on the thickness of the fat and cholesterol comprising the plaque. The present invention utilizes this phenomenon to measure the depth of plaque on the inner walls of blood vessels.

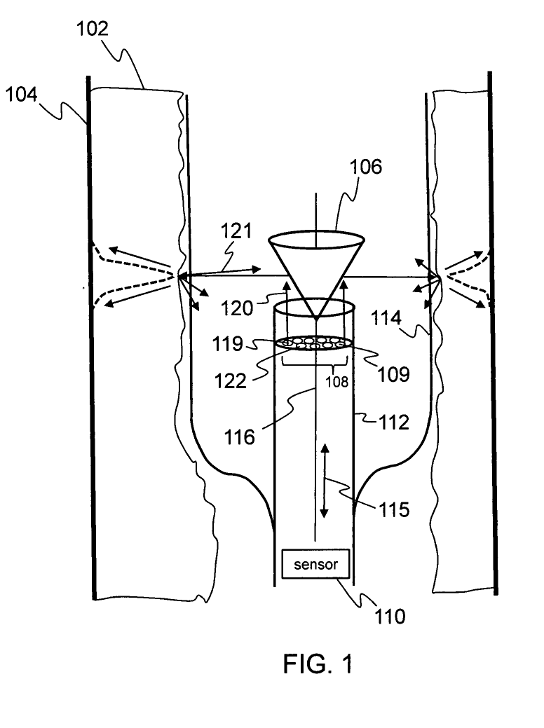

FIG. 1 is an illustration showing an imaging device 100 in accordance with the present invention situated within a blood vessel having fat and cholesterol plaque 102 on the inner vessel walls 104. Imaging components including a mirror 106, fiber optic bundle 108 having a terminus 109, and sensor 110 reside inside a catheter tube 112 having a transparent catheter balloon 114 which expands to meet the plaque 102 surface. In a desired aspect, the imaging components are capable of moving axially 115 within the catheter tube 112. In a further desired aspect, the mirror 106 is substantially conical in shape, although any mirror shape capable of performing the reflection needs of the system may be used. The conical mirror 106 is held at an apex by a holder 116 at the center of the fiber optic bundle 108. The holder 116 can be independent or connected with a guide wire of the catheter. A projection portion 119 of the fiber optic bundle 108 are used to project several narrow band laser beam dots 120 around the center, which are then reflected by the conical mirror 106 and transmitted through the transparent balloon 114 to the plaque tissues 102 on the vessel walls 104. The projected laser dots 120 on the plaques 102 are reflected back as an image signal 121 to the fiber optic bundle 108, where a receiving portion 122 of the fiber optic bundle 108 receives and transmits the reflected image signal 121 to the sensor 110. The sensor 110 can be anything known in the art for capturing a light signal, including a camera, charge coupled device (CCD), diode, or photo cell. A non-coherent portion of the reflected image signal 121 is expected to have a substantially Gaussian profile. The shape of the Gaussian profile or a best fit curve of the diffusive image intensity of the reflected laser dot varies with respect to the depth of the plaque. Therefore, the depth of the plaque on the blood vessel can be resolved by measuring the laser dot profile. This method works on the non-coherent portions of the reflected light, or it can be used in conjunction with optical coherence tomography.

It should be noted that other research, as described in U.S. Patent Publication No. US 2005/0251116 A1, incorporated by reference as though fully disclosed herein, uses a prism coupled with a mechanical rotation device to obtain images of the surrounding tissues from all angles. This system, however, will cause major challenges in packaging due to the relative large size of the mechanical equipment. In contrast, since the small conical mirror of the present invention can reflect the laser beam to all angles of the surrounding tissues as well as direct the images from all angles back to the fiber bundle, the rotating mechanical equipment of the above cited publication is not required. Furthermore, the configuration of the present invention allows the entire plaque surface surrounding the balloon catheter to be measured simultaneously.

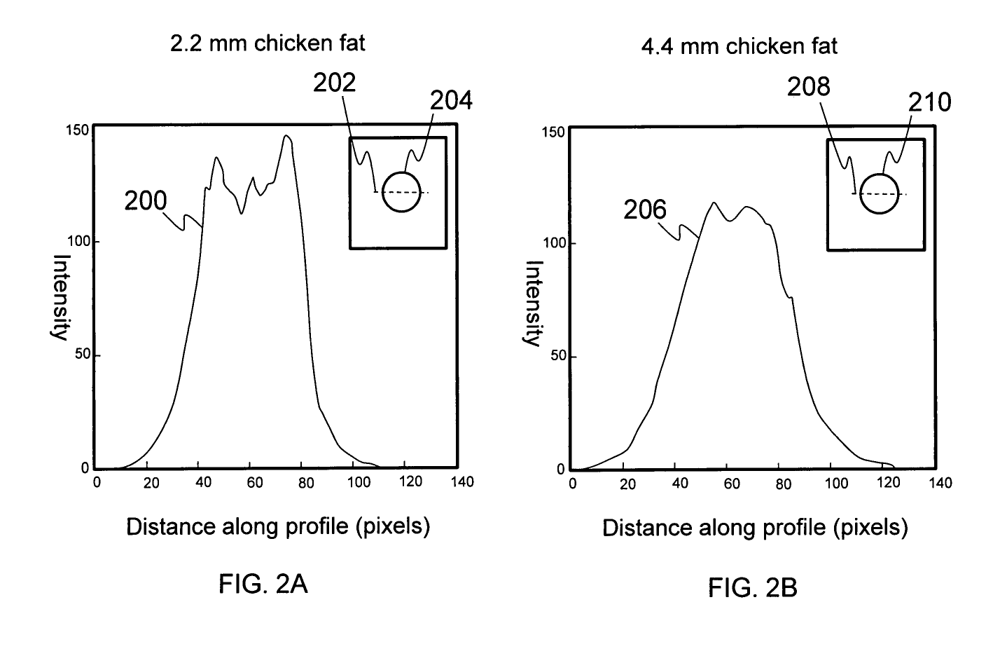

FIG. 2A shows the Gaussian profile 200 of a cross section 202 of a diffusive laser dot image 204 on a chicken fat surface with a depth of 2.2 mm. The diameter of the Gaussian profile 200 is 100 pixels. Similarly, FIG. 2B shows the Gaussian profile 206 of a cross section 208 of a diffusive laser dot image 204 on a thicker chicken fat surface with a depth of 4.4 mm. The diameter of the Gaussian profile is 120 pixels. As can be seen from the graphs in FIGS. 2A and 2B, the diameter of the diffusive dot image and therefore width of the Gaussian profile increases with the depth of the fat layer.

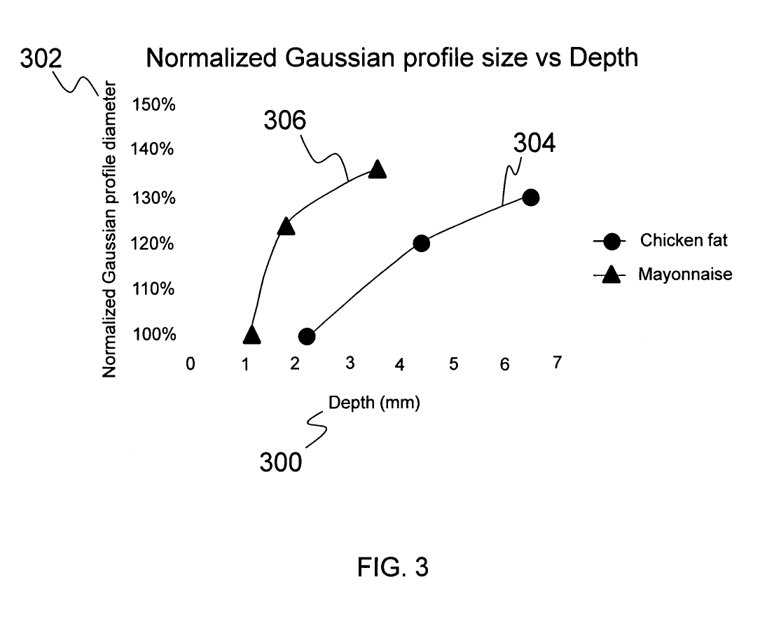

A normalized graph of Gaussian profiles at various depths can be constructed using experimental data as shown in FIG. 3. The graph in FIG. 3 plots fat layer depth 300 against normalized Gaussian profile diameter 302. The graph contains two best fit curves, one for a series of data points taken for chicken fat 304, and one for a series of data points taken for mayonnaise 306. A similar best fit curve can be constructed for a layer of human fat and cholesterol as found in the plaques on blood vessel walls. When a patient is tested using the catheter imaging system of the present invention, the Gaussian profile obtained is compared to a best-fit curve in a normalized graph as in FIG. 3 to determine the depth of the plaque on the patient's vessel walls.

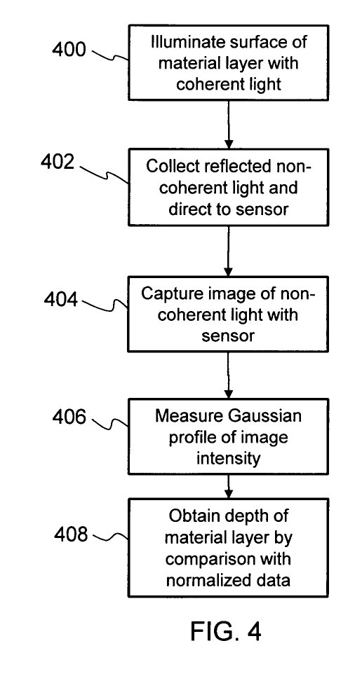

The present invention also comprises the general method of obtaining depth information of material coating from an intensity profile of reflected light. The acts in the method are illustrated in FIG. 4. The first act is illuminating 400 the surface of a material layer with coherent light. The material layer may be a layer of blood vessel plaque, but the method is generally applicable to any material layer which will produce a reflected image having a substantially Gaussian intensity distribution. The light reflected from the material layer will be non-coherent and have a substantially Gaussian profile. The non-coherent light is then collected 402 and directed to a sensor where the non-coherent light image is captured 404 by the sensor. The length of a diametrical profile of the reflected non-coherent image is then measured 406 and compared with previously obtained normalized graphical data representing the relationship between profile diameter and material layer depth (as in FIG. 3), whereby the depth of the material layer is obtained 408.

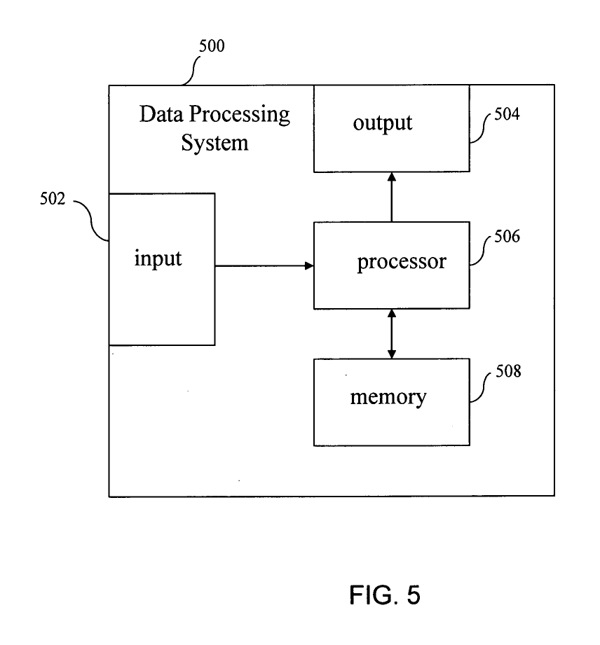

As can be appreciated by one skilled in the art, the present invention also comprises a data processing system for executing the method of the present invention, as previously mentioned. A block diagram depicting the components of an image processing system of the present invention is provided in FIG. 5. The image processing system 500 comprises an input 502 for receiving information from at least one sensor for use in detecting image intensity of the non-coherent light captured by the sensor. Note that the input 502 may include multiple “ports.” Typically, input is received from at least one sensor, non-limiting examples of which include video image sensors. An output 504 is connected with the processor for providing information regarding the intensity profile of the image to other systems in order that a network of computer systems may serve as an image processing system. Output may also be provided to other devices or other programs; e.g., to other software modules, for use therein. The input 502 and the output 504 are both coupled with a processor 506, which may be a general-purpose computer processor or a specialized processor designed specifically for use with the present invention. The processor 506 is coupled with a memory 508 to permit storage of data and software that are to be manipulated by commands to the processor 506.

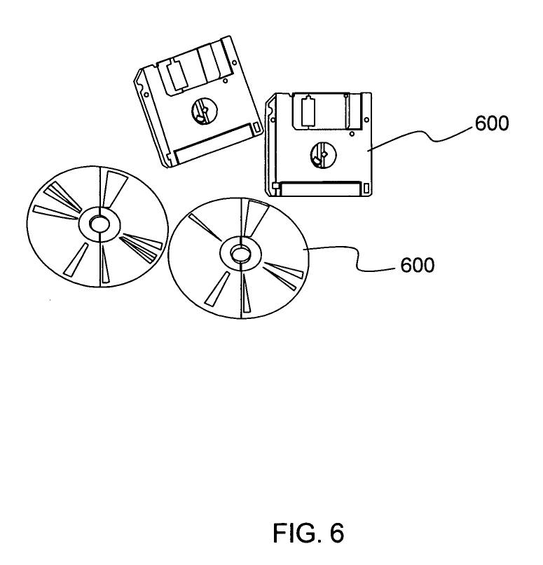

The present invention also comprises a computer program product. An illustrative diagram of a computer program product embodying the present invention is depicted in FIG. 6. The computer program product 600 is depicted as an optical disk such as a CD or DVD. However, as mentioned previously, the computer program product generally represents computer-readable instruction means stored on any compatible computer-readable medium. The term “instruction means” as used with respect to this invention generally indicates a set of operations to be performed on a computer, and may represent pieces of a whole program or individual, separable, software modules. Non-limiting examples of “instruction means” include computer program code (source or object code) and “hard-coded” electronics (i.e. computer operations coded into a computer chip). The “instruction means” may be stored in the memory of a computer or on a computer-readable medium such as a floppy disk, a CD-ROM, and a flash drive.

What is claimed is:

1. A device for measuring the depth of a material layer, comprising:
a fiber optic bundle extending along an axis and comprising a projection portion and a receiving portion; where
the projection portion is configured to project light onto a material layer surface; and
the receiving portion is configured to receive a reflected image signal of the projected light from the material layer;
a mirror positioned at a terminus of the fiber optic bundle, the mirror configured to:
reflect projected light from the projection portion of the fiber optic bundle at an angle substantially perpendicular to the axis of the fiber optic bundle to illuminate the material surface; and
reflect the reflected image signal of the projected light from the material layer at an angle substantially perpendicular to the axis of the fiber optic bundle, such that the reflected image signal can be received by the receiving portion of the fiber optic bundle; and
a sensor configured to receive the reflected image signal from the receiving portion of the fiber optic bundle, whereby the image signal can be analyzed to determine the depth of the material layer.

2. The device of claim 1, where the fiber optic bundle is mounted within a balloon catheter.

3. The device of claim 2, wherein the device is configured to move axially within the balloon catheter.

4. The device of claim 3, where the mirror is held by a holder portion near a center of the fiber optic bundle.

5. The device of claim 4, where the mirror is substantially conical in shape, and positioned such that an apex of the conical mirror is located proximal to the terminus of the fiber optic bundle.

6. The device of claim 1, where the mirror is held by a holder portion near a center of the fiber optic bundle.

7. The device of claim 1, where the mirror is substantially conical in shape, and positioned such that an apex of the conical mirror is located proximal to the terminus of the fiber optic bundle.

8. A method for determining a depth of a material layer, comprising acts of:
projecting light onto a surface of the material layer;
receiving a reflected image signal of the projected light from the material layer surface;
capturing the reflected image signal with a sensor;
measuring an image intensity profile of the captured image; and
determining the depth of the material layer by comparison of the measured image intensity profile with a pre-obtained normalized data set.

9. The method of claim 8, wherein the acts of projecting, receiving, and capturing are executed using a catheter imaging system, the catheter imaging system comprising:
a fiber optic bundle extending along an axis and comprising a projection portion and a receiving portion; where
the projection portion is configured to project light onto a material layer surface; and
the receiving portion is configured to receive a reflected image signal of the projected light from the material layer;
a mirror positioned at a terminus of the fiber optic bundle, the mirror configured to:
reflect projected light from the projection portion of the fiber optic bundle at an angle substantially perpendicular to the axis of the fiber optic bundle to illuminate the material surface; and
reflect the reflected image signal of the projected light from the material layer at an angle substantially perpendicular to the axis of the fiber optic bundle, such that the reflected image signal can be received by the receiving portion of the fiber optic bundle; and
a sensor configured to receive the reflected image signal from the receiving portion of the fiber optic bundle.

10. The method of claim 9, where the mirror is substantially conical in shape, and positioned such that an apex of the conical mirror is located proximal to the terminus of the fiber optic bundle.

11. A data processing system for measuring the depth of a material layer, comprising one or more processors configured to cause the system to perform operations of:
projecting light onto a surface of the material layer;
receiving a reflected image signal of the projected light from the material layer surface;
capturing the reflected image signal with a sensor;
measuring an image intensity profile of the captured image; and
determining the depth of the material layer by comparison of the measured image intensity profile with a pre-obtained normalized data set.

12. The data processing system of claim 11, wherein the operations of projecting, receiving, and capturing are executed using a catheter imaging system, the catheter imaging system comprising:
a fiber optic bundle extending along an axis and comprising a projection portion and a receiving portion; where
the projection portion is configured to project light onto a material layer surface; and
the receiving portion is configured to receive a reflected image signal of the projected light from the material layer;
a mirror positioned at a terminus of the fiber optic bundle, the mirror configured to:
reflect projected light from the projection portion of the fiber optic bundle at an angle substantially perpendicular to the axis of the fiber optic bundle to illuminate the material surface; and
reflect the reflected image signal of the projected light from the material layer at an angle substantially perpendicular to the axis of the fiber optic bundle, such that the reflected image signal can be received by the receiving portion of the fiber optic bundle; and
a sensor configured to receive the reflected image signal from the receiving portion of the fiber optic bundle.

13. The data processing system of claim 12, where the mirror is substantially conical in shape, and positioned such that an apex of the conical mirror is located proximal to the terminus of the fiber optic bundle.

14. A computer program product for measuring the depth of a material layer, comprising computer instruction means encoded on a computer-readable medium executable by a computer having a processor for causing an imaging system to perform operations of:
projecting light onto a surface of the material layer;
receiving a reflected image signal of the projected light from the material layer surface;
capturing the reflected image signal with a sensor;
measuring an image intensity profile of the captured image; and
determining the depth of the material layer by comparison of the measured image intensity profile with a pre-obtained normalized data set.

15. The computer program product of claim 14, wherein the operations of projecting, receiving, and capturing are executed using a catheter imaging system, the catheter imaging system comprising:
a fiber optic bundle extending along an axis and comprising a projection portion and a receiving portion; where
the projection portion is configured to project light onto a material layer surface; and
the receiving portion is configured to receive a reflected image signal of the projected light from the material layer;
a mirror positioned at a terminus of the fiber optic bundle, the mirror configured to:
reflect projected light from the projection portion of the fiber optic bundle at an angle substantially perpendicular to the axis of the fiber optic bundle to illuminate the material surface; and
reflect the reflected image signal of the projected light from the material layer at an angle substantially perpendicular to the axis of the fiber optic bundle, such that the reflected image signal can be received by the receiving portion of the fiber optic bundle; and
a sensor configured to receive the reflected image signal from the receiving portion of the fiber optic bundle.

16. The computer program product of claim 15, where the mirror is substantially conical in shape, and positioned such that an apex of the conical mirror is located proximal to the terminus of the fiber optic bundle.
